(19) United States
(12) Patent Application Publication
(10) Pub. No.: US 2025/0065133 A1
(43) Pub. Date: Feb. 27, 2025

(54) IMPLANT COMMUNICATION SYSTEM AND METHOD FOR COMMUNICATING WITH AN IMPLANTABLE MEDICAL DEVICE

(71) Applicant: BIOTRONIK SE & Co. KG, Berlin (DE)

(72) Inventors: Hanno WUNDERLICH, Berlin (DE); Enrico STEPHAN, Berlin (DE); Thomas KRUCZYK, Berlin (DE); Volker OTTO, Berlin (DE); Pauline GLODOWSKI, Berlin (DE); Xenia GEHRKE, Berlin (DE); Carsten HENNIG, Berlin (DE); Maximilian LOEW, Berlin (DE); Steven HERRSCHAFT, Berlin (DE); Rebecca JOST, Berlin (DE)

(73) Assignee: BIOTRONIK SE & Co. KG, Berlin (DE)

(21) Appl. No.: 18/725,187

(22) PCT Filed: Dec. 15, 2022
(86) PCT No.: PCT/EP2022/086124

§ 371 Date: Jun. 28, 2024

(30) Foreign Application Priority Data
Jan. 14, 2022 (EP) 22151560.4

(51) Int. Cl. A61N 1/372; G06F 9/455; G16H 40/67

(52) U.S. Cl. CPC A61N 1/37252; A61N 1/37235; G06F 9/45558; G16H 40/67; G06F 2009/45595

(57) ABSTRACT

An implant communication system configured to receive the data transferred by the first control unit and to simulate the communication interface, the back-end communication unit further comprising a programmer app unit configured to read out data from the communication interface simulation unit, the back-end communication unit further comprising a second control unit configured to control the communication interface simulation unit and the programmer app unit in such a way that the data is saved, in particular as a report, in a storage unit communicatively connected to the second control unit and/or the back-end communication unit, said data being accessible by the health care provider. Furthermore, the invention relates to a computer implemented method for communicating with an implantable medical device and a computer program.

### CROSS-REFERENCE TO RELATED APPLICATIONS

This application is the United States National Phase under 35 U.S.C. § 371 of PCT International Patent Application No. PCT/EP2022/086124, filed on Dec. 15, 2022, which claims the benefit of European Patent Application No. 22151560.4, filed on Jan. 14, 2022, the disclosures of which are hereby incorporated by reference herein in their entireties.

### TECHNICAL FIELD

The present invention relates to an implant communication system. Furthermore, the present invention relates to a computer implemented method for communicating with an implantable medical device.

### BACKGROUND

U.S. Publication No. 2020/0357513 A1 discloses techniques for remote monitoring of a patient and corresponding medical device(s). The remote monitoring comprises providing an interactive session configured to allow a user to navigate a plurality of sub sessions, determining a first set of data items in accordance with a first sub session, the first set of data items including the image data, determining a second set of data items in accordance with a second sub session of the interactive session, determining, based at least in part on the first set of data items and the second set of data items, an abnormality, and outputting a post-implant report of the interactive session.

International Publication No. 2007/070669 A2 discloses a patient management device for portably interfacing with a plurality of implantable medical devices and method thereof is presented. Permission to interrogate one or more implantable medical devices is authenticated. Patient device data is individually exchanged through interrogation of at least one authenticated implantable medical device through short range telemetry. External device data is exchanged via communication with at least one external device through long range telemetry. At least one of the patient device and external device data is maintained contemporaneously to execution of operations to perform one or more of relay, processing, and outputting of the patient device and external device data subsequent to the interrogation of the implantable medical device.

Conventional implant systems hence comprise a programmer capable of reading out data from and writing data to an implantable medical device. Such proprietary programming devices can also be used solely for reading out data from the implantable medical device.

If a patient wearing an implantable medical device is admitted to a hospital and/or medical practice due to an acute medical condition data is read out from the implantable medical device by means of said programmer by a trained expert and/or medical practitioner in order to evaluate medical parameters of the patient recorded by the implantable medical device as well as technical parameters of the implantable medical device in order to evaluate if the implantable medical device is operating normally or whether a technical issue is present.

Programmers however are not universally available at all medical facilities such as hospitals and/or medical practices. Furthermore, such programmers may be difficult to use for untrained medical staff, acceptance of such devices is generally limited. In addition, programming devices are equipped with many functions which, if used incorrectly, can adversely affect the health of the patient/implant carrier.

The present disclosure is directed toward overcoming one or more of the above-mentioned problems, though not necessarily limited to embodiments that do.

### SUMMARY

It is therefore an object of the present invention to provide an improved implant communication system that is usable not only by medical practitioners but also by untrained medical staff and is generally widely accepted and universally available.

at least the object is solved by an implant communication system having the features of claim 1.

Furthermore, at least the object is solved by a computer implemented method for communicating with an implantable medical device having the features of claim 14.

In addition, at least the object is solved by a computer program with program code to perform the method according to the present invention when the computer program is executed on a computer having the features of claim 15.

Further developments and advantageous embodiments are defined in the dependent claims.

The present invention provides an implant communication system, comprising a front-end communication unit comprising a communication interface configured to read out data from the implantable medical device, and a first control unit communicatively connected to the communication interface, the first control unit being configured to transfer the data read out by the communication interface to a back-end communication unit.

The system further comprises the back-end communication unit comprising a communication interface simulation unit configured to receive the data transferred by the first control unit and to simulate the communication interface, the back-end communication unit further comprising a programmer app unit configured to read out data from the communication interface simulation unit, the back-end communication unit further comprising a second control unit configured to control the communication interface simulation unit and the programmer app unit in such a way that the data is saved, in particular as a report, in a storage unit communicatively connected to the second control unit and/or the back-end communication unit, said data being accessible by the health care provider.

Furthermore, the present invention provides a computer implemented method for communicating with an implantable medical device. The method comprises reading out data from the implantable medical device by means of a front-end communication unit comprising a communication interface and a first control unit communicatively connected to the communication interface and transferring the data read out by the communication interface to a back-end communication unit by means of the first control unit.

Furthermore, the method comprises receiving the data transferred by the first control unit by means of a communication interface simulation unit comprised by the back-end communication unit and simulating the communication interface by means of the communication interface simulation unit.

In addition, the method comprises reading out data from the communication interface simulation unit by means of a programmer app unit comprised by the back-end communication unit, and controlling the communication interface simulation unit and the programmer app unit by means of a second control unit comprised by the back-end communication unit in such a way that the data is saved, in particular as a report, in a storage unit communicatively connected to the second control unit and/or the back-end communication unit, said data being accessible by the health care provider.

An idea of the present invention is to provide medical staff with an easy to use and widely available tool for accessing a patient's implantable medical device for the purposes of reading out data related to a condition of the patient, i.e., medical and/or technical parameters recorded by the implantable medical device.

Moreover, the system enables processing of implant data in a backend, and giving the customer or untrained specialist staff only a simple device. The system thus enables the user to carry out implant queries at various points of care, e.g., emergency room, hospital bed, nursing home without the presence of staff trained for programming devices. The present invention hence enables a reduction of on-site service support by field staff and thereby a reduction of operating costs, as well as a reduction of waiting times and thus an increase in efficiency in patient management.

The implantable medical device may be formed by a purely therapeutic implant and/or an implant with therapeutic and diagnostic functions.

An example of a purely therapeutic implant/implantable medical device is, e.g., a stimulator/electrode for deep brain stimulation. The therapy consists of the delivery of pulse trains without collecting diagnostic data from the stimulator.

An example of a purely diagnostic implant is, e.g., a cardiac rhythm monitor. The diagnostic function consists of continuous recording of the patient's ECG and automatic evaluation of abnormalities of the heart rhythm. If such are detected, an ECG recording is stored and typically automatically transmitted to a remote monitoring system.

An example of an implant with therapeutic and diagnostic functions is, e.g., a cardiac pacemaker. The pacemaker is typically implanted subcutaneously in the upper right thoracic region and the electrode is placed in the patient's heart via a large vein. The therapeutic function consists of delivering stimulation pulses to trigger a cardiac action, provided there is no spontaneous cardiac action in the patient. The diagnostic function consists, for example, in the continuous recording of the patient's ECG and automatic evaluation of abnormalities of the heart rhythm. If such are detected, an ECG recording is stored and typically automatically transmitted to a remote monitoring system.

According to an aspect of the present invention, the data is transferred from the first control unit to a cloud server, wherein a cloud gateway is configured to fetch the data from the cloud server and store it, wherein the cloud gateway is further configured to make the data available for retrieval by a dispatch unit of the back-end communication unit.

The cloud server serves as short-term storage for the data to be transferred until the data is collected and stored by the cloud gateway. The cloud server is operated by a cloud hoster, whereas the cloud gateway is operated in a proprietary infrastructure. The cloud gateway represents the long-term storage for the data and forwards it to the requesting systems, e.g., the dispatch unit.

According to a further aspect of the present invention, the back-end communication unit comprises a plurality of report creators (also called report generators RG), each report creator comprising a version of the communication interface simulation unit, the programmer app unit and the second control unit, wherein the dispatch unit is configured to check at predetermined intervals for new data in the cloud gateway, and is further configured to download and process said data, and during processing, to use metadata to determine to which report creator the data is sent to.

Basically, the following applies to report generators RGs: a) there should be several RGs: b) the transmitted implant data should be used to decide which RG is to be used: c) the decision is based on: implant type, if necessary also on implant version and/or approval region: and d) further RG instances should be available for load distribution.

For example, the following embodiment is possible: First, a decision is made as to which program version is required. The programmer version should always or usually be the latest release in the approval region (e.g., in US, CE). This release combines all implant applications. When interrogating the transmitted data, the programmer branches to the associated application or RG and generates the PDF and/or report. As an alternative or in addition, it may also happen that a special programmer version is required that differs from the latest release because it is a special version (e.g., firmware version) of the implant.

The report generator or report creator is available in different versions or designs. This is because the data has a certain format according to the firmware version of the implantable medical device. Thus, the data can only be processed by certain versions of the programmer software. The dispatch unit therefore selects the report creator on which the correct programmer software is running. For this purpose, the dispatch unit also uses information on released programmer software versions from a release tool. The release tool provides the information on released software versions in, e.g., an XML file. This can be read by the dispatch unit to decide which is the currently valid programmer software and/or report creator for a country.

According to a further aspect of the present invention, the metadata comprises an implant serial number, an implant type and/or an implant firmware version, wherein the respective report creator is configured to process data having a predetermined format matching the firmware version of the implantable medical device. Said metadata thus advantageously enables to identify the correct report creator.

According to a further aspect of the present invention, each report creator is a virtual machine, and wherein the communication interface simulation unit, the programmer app unit and the second control unit are applications installed in the virtual machine. Thus, the communication interface simulation unit is advantageously capable of simulating the front-end communication unit and/or the connection between the communication interface and the implantable medical device. The communication interface simulation unit may act as a usual programming head for the programmer software that speaks directly to the implant. In other words, the communication interface simulation unit makes it appear to the programmer software as if it were communicating directly with the implant via a connected programming head (as would be the case with the real programmer hardware).

The second control unit controls the communication interface simulation unit and the programmer app unit in such a way that the data from the implantable medical device is evaluated and saved as a report document in the storage unit. This data is then taken over again by the dispatch unit and made available to the health care provider and/or by an intermediary security entity.

According to a further aspect of the present invention, the communication interface is a reading and/or programming head configured to establish a connection to the implantable medical device, and wherein the data acquired by an implantable medical device is cardiac current curve data, IEGM live recordings (e.g., over 30 seconds), information on the condition of components (e.g., battery, electrodes, etc.) and/or statistical values on recorded episodes. Thus, advantageously any existing communication interface can be used to establish the connection to the implantable medical device.

According to a further aspect of the present invention, the data is encrypted by the first control unit, and wherein the data fetched from the cloud gateway is decrypted using a certificate and key management system located in the back-end communication unit. The data to be transmitted may be encrypted end-to-end between the first control unit and the dispatch unit. In addition to this end-to-end encryption, there is also transport encryption between the first control unit and the cloud server, between the cloud server and the cloud gateway, and/or between the cloud gateway and the dispatch unit.

The time server is basically required so that the current time can be obtained (from the overall system and/or from each individual component, e.g., the first control unit). The current time obtained from the time server may be necessary to be able to set up the transport encryption between the first control unit and the cloud server, and thus for establishing a secure connection to the back-end.

According to a further aspect of the present invention, the data may be encrypted by the first control unit using a time server and/or using time data from the time server.

According to a further aspect of the present invention, information generated from the data stored in the storage unit is available for retrieval via a web application, wherein the data is accessible by the health care provider and/or by an intermediary security entity. Hence, the health care provider can advantageously be informed of the medical condition of the patient based on the data acquired by the implantable medical device within a short period of time.

For reasons of data protection, the route via the intermediary security instance is preferred. In one embodiment, the clinical staff is first contacted by a customer support, e.g., via telephone and asked to provide identification information of staff and device. The ident information is preferably contact information for the clinical staff and a session ID, e.g., the serial number of the implant, which appears on a display of the front-end communication unit. This process takes place in parallel with the data transfer and/or processing of the implant data in the backend.

Alternatively or additionally, the health care provider PR (also called user or clinical staff) in the clinic calls first the customer support SP after a successful data request and sends contact information and the session ID (shown on a display of the front-end communication unit) to the SP. The SP then forwards all this information (including the session ID) to the responsible sales representative, an intermediary security entity IN, who/which then contacts the user PR. In other words, based on the ident info (e.g., session ID and/or contact information), the customer support SP then identifies the responsible security instance IN, e.g., a specific sales representative. This IN then receives the necessary information, e.g., via SMS, to retrieve the information generated from the implant data and forward it to the clinical staff.

The session ID is generated before the data is transmitted in the first control unit, e.g., with the help of the implant serial number, the device serial number and/or the current time. Customer Support then identifies the responsible intermediary security entity and sends a message, e.g., an SMS, with the relevant information. The intermediary security entity, e.g., a company representative, then looks for the report document with the correct session ID in the web application analyses it and then reports to the health care provider.

According to a further aspect of the present invention, the first control unit comprises a communication logic configured to communicate with different implantable medical devices in order to query identification data of the implant, in particular a model and/or firmware version, wherein the first control unit is further configured, based on the identification data, to read predetermined memory areas of the implantable medical device. The customer or untrained specialist personnel are thus given a simple device. This device is configured to interact with both the implant and the back-end.

According to a further aspect of the present invention, information contained in the predetermined memory areas of the implantable medical device comprises a current programming of the implantable medical device, trend data of the last 24 hours, last measured values from automatic tests, long-term trend data, a list of stored episodes, episode details of the episodes within the last 24 hours, patient data stored in the implant and/or a predetermined interval of live IEGM data. Based on said data, a comprehensive report on a patient medical history can be compiled.

According to a further aspect of the present invention, the front-end communication unit is configured to send metadata from the implantable medical device to the back-end communication unit, wherein the back-end communication unit is configured to determine which memory areas the front-end communication unit is to query, and wherein the back-end communication unit is configured to transmit information on said memory areas to the front-end communication unit. Thus, the software operating on the front-end communication unit is not required to be updated with each new implant model or implant firmware so that it can interrogate the implants.

According to a further aspect of the present invention, the cloud gateway is configured to distribute software updates to the cloud server, wherein the front-end communication unit is configured to query the cloud server for software updates. If an update is available, the front-end communication unit can then decide independently whether to download and install the update or not. Further, the cloud gateway forwards a predetermined distribution rule to the cloud server. On of devices (e.g., the first control unit), the cloud server then checks whether an update is available for the distribution rule. Thus, advantageously the front-end communication unit is configured to automatically receive software updates according to said predetermined distribution rule.

According to a further aspect of the present invention, the cloud server is configured to check, preferably on request of the first control unit and/or front-end communication unit, whether there is a distribution rule for the front-end communication unit, in particular based on a serial number and/or country/region of the front-end communication unit, wherein if a software update is available, the cloud server is configured to notify the front-end communication unit about the update. Hence, a suitable version of the software update can advantageously be determined for each individual front-end communication unit.

The herein described features of the system for providing data acquired by an implantable medical device to a health care provider are also disclosed for the computer implemented method for providing data acquired by an implantable medical device to a health care provider and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a diagram of an implant communication system according to a preferred embodiment of the present invention: and

FIG. 2 shows a flowchart of a computer implemented method for communicating with an implantable medical device according to the preferred embodiment of the present invention.

### DETAILED DESCRIPTION

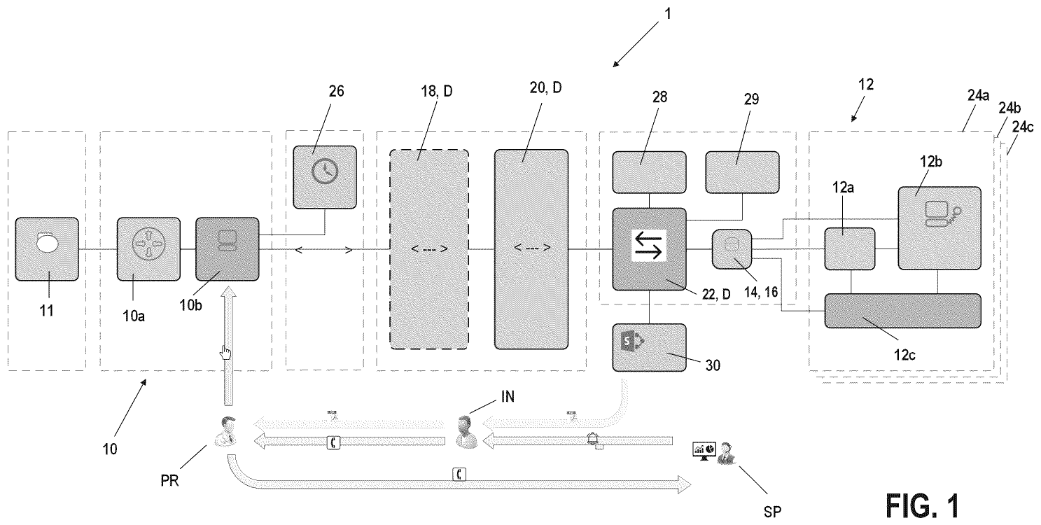

The implant communication system 1 of FIG. 1 comprises a front-end communication unit 10 comprising a communication interface 10a configured to read out data D from an implantable medical device 11, and a first control unit 10b communicatively connected to the communication interface 10a, the first control unit 10b being configured to transfer the data D read out by the communication interface 10a to a back-end communication unit 12.

Furthermore, the implant communication system 1 comprises the back-end communication unit 12 comprising a communication interface simulation unit 12a configured to receive the data D transferred by the first control unit 10b and to simulate the communication interface 10a, the back-end communication unit 12 further comprising a programmer app unit 12b configured to read out data D from the communication interface simulation unit 12a.

In addition, the back-end communication unit 12 comprises a second control unit 12c configured to control the communication interface simulation unit 12a and the programmer app unit 12b in such a way that the data D is saved, in particular as a report 14, in a storage unit 16 communicatively connected to the second control unit 12c and/or the back-end communication unit 12, said data D being accessible by the health care provider PR.

The data D is transferred from the first control unit 10b to a cloud server 18, wherein a cloud gateway 20 is configured to fetch the data D from the cloud server 18 and store it, wherein the cloud gateway 20 is further configured to make the data D available for retrieval by a dispatch unit 22 of the back-end communication unit 12.

The back-end communication unit 12 comprises a plurality of report creators 24a, 24b, 24c, each report creator 24a, 24b, 24c comprising a version of the communication interface simulation unit 12a, the programmer app unit 12b and the second control unit 12c. The dispatch unit 22 is further configured to check at predetermined intervals for new data D in the cloud gateway 20, and is further configured to download and process said data D, and during processing, to use metadata MD to determine to which report creator 24a, 24b, 24c the data D is sent to.

The metadata MD comprises an implant serial number, an implant type and/or an implant firmware version. Furthermore, the respective report creator 24a, 24b, 24c is configured to process data D having a predetermined format matching the firmware version of the implantable medical device 11.

Each report creator 24a, 24b, 24c is a virtual machine, and wherein the communication interface simulation unit 12a, the programmer app unit 12b and the second control unit 12c are applications installed in the virtual machine.

The communication interface 10a is a reading and/or programming head configured to establish a connection to the implantable medical device 11. Moreover, the data D acquired by an implantable medical device 11 is cardiac current curve data D. The data D is encrypted by the first control unit 10b, and wherein the data D fetched from the cloud gateway 20 is decrypted using a certificate and key management system 28 located in the back-end communication unit 12. In this embodiment, it is further necessary to obtain the current time from the time server 26 to set up a transport encryption between the first control unit 10b and the cloud server 18.

Information generated from the data D stored in the storage unit 16 is available for retrieval via a web application 30, wherein the data D is accessible by the health care provider PR and/or by an intermediary security entity IN.

In parallel to this transfer and the backend activities, the user can, e.g., call “Customer Support” SP in the “Local User” clinic and give their contact information, as well as a session ID, which will be shown on a display of the front-end communication unit.

The first control unit 10b contains a communication logic configured to communicate with different implantable medical devices 11 in order to query identification data D of the implantable medical device 11, in particular a model and/or firmware version. The first control unit 10b is further configured, based on the identification data D, to read predetermined memory areas of the implantable medical device 11.

Information contained in the predetermined memory areas of the implantable medical device 11 comprises a current programming of the implantable medical device 11, trend data D of the last 24 hours, last measured values from automatic tests, long-term trend data D, a list of stored episodes, episode details of the episodes within the last 24 hours, patient data D stored in the implant and/or a predetermined interval of live IEGM data D.

According to a further embodiment, the front-end communication unit 10 is configured to send metadata MD from the implantable medical device 11 to the back-end communication unit 12. The back-end communication unit 12 is then configured to determine which memory areas the front-end communication unit 10 is to query.

In addition, the back-end communication unit 12 is configured to transmit information on said memory areas to the front-end communication unit 10. The cloud gateway 20 is configured to distribute software updates to the cloud server 18, wherein the front-end communication unit 10 is configured to query the cloud server 18 for software updates.

The cloud server 18 is here configured to check on request of the front-end communication unit (here in particular the control unit 10b) whether there is a distribution rule for the front-end communication unit 10, in particular based on a serial number and/or country/region of the front-end communication unit 10, wherein if a software update is available, the cloud server 18 is configured to notify the front-end communication unit 10 about the update.

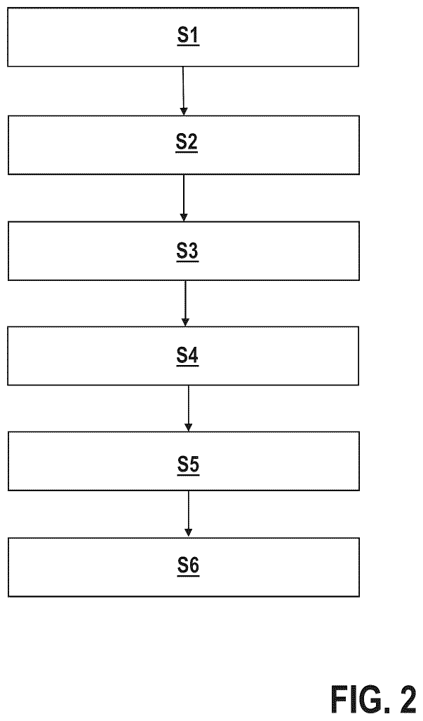

FIG. 2 shows a flowchart of a computer implemented method for communicating with an implantable medical device 11 according to the preferred embodiment of the present invention. The method comprises reading out S1 data D from the implantable medical device 11 by means of a front-end communication unit 10 comprising a communication interface 10a and a first control unit 10b communicatively connected to the communication interface 10a and transferring S2 the data D read out by the communication interface 10a to a back-end communication unit 12 by means of the first control unit 10b.

Moreover, the method comprises receiving S3 the data D transferred by the first control unit 10b by means of a communication interface simulation unit 12a comprised by the back-end communication unit 12 and simulating S4 the communication interface 10a by means of the communication interface simulation unit 12a.

In addition, the method comprises reading out S5 data D from the communication interface simulation unit 12a by means of a programmer app unit 12b comprised by the back-end communication unit 12, and controlling S6 the communication interface simulation unit 12a and the programmer app unit 12b by means of a second control unit 12c comprised by the back-end communication unit 12 in such a way that the data D is saved, in particular as a report, in a storage unit 16 communicatively connected to the second control unit 12c and/or the back-end communication unit 12, said data D being accessible by the health care provider PR.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 system
10 front-end communication unit
10a communication interface
10b first control unit
11 implantable medical device
12 back-end communication unit
12a communication interface simulation unit
12b programmer app
12c second control unit
14 report
16 storage unit
18 cloud server
20 cloud gateway
22 dispatch unit
24a, 24b, 24c report creators
26 time server
28 certificate and key management system
30 web application
D data
IN intermediary security entity
MD metadata
PR health care provider
SP customer support
S1-S6 method steps

1. Implant communication system, comprising:
a front-end communication unit comprising a communication interface configured to read out data from an implantable medical device, and a first control unit communicatively connected to the communication interface, the first control unit being configured to transfer the data read out by the communication interface to a back-end communication unit; and
the back-end communication unit comprising a communication interface simulation unit configured to receive the data transferred by the first control unit and to simulate the communication interface, the back-end communication unit further comprising a programmer app unit configured to read out data from the communication interface simulation unit, the back-end communication unit further comprising a second control unit configured to control the communication interface simulation unit and the programmer app unit in such a way that the data is saved, in particular as a report, in a storage unit communicatively connected to the second control unit and/or the back-end communication unit, said data being accessible by a health care provider.

2. System of claim 1, wherein the data is transferred from the first control unit to a cloud server, wherein a cloud gateway is configured to fetch the data from the cloud server and store it, wherein the cloud gateway is further configured to make the data available for retrieval by a dispatch unit of the back-end communication unit.

3. System of claim 2, wherein the back-end communication unit comprises a plurality of report creators, each report creator comprising a version of the communication interface simulation unit, the programmer app unit and the second control unit, wherein the dispatch unit is configured to check at predetermined intervals for new data in the cloud gateway, and is further configured to download and process said data, and during processing, to use metadata to determine to which report creator the data is sent to.

4. System of claim 3, wherein the metadata comprises an implant serial number, an implant type and/or an implant firmware version, wherein the respective report creator is configured to process data having a predetermined format matching the firmware version of the implantable medical device.

5. System of claim 3, wherein each report creator is a virtual machine, and wherein the communication interface simulation unit, the programmer app unit and the second control unit are applications installed in the virtual machine.

6. System of claim 1, wherein the communication interface is a reading and/or programming head configured to establish a connection to the implantable medical device, and wherein the data acquired by an implantable medical device is cardiac current curve data.

7. System of claim 1, wherein the data is encrypted by the first control unit, and wherein the data fetched from the cloud gateway is decrypted using a certificate and key management system located in the back-end communication unit.

8. System of claim 1, wherein information generated from the data stored in the storage unit is available for retrieval via a web application, wherein the data is accessible by the health care provider and/or by an intermediary security entity IN.

9. System of claim 1, wherein the first control unit contains a communication logic configured to communicate with different implantable medical devices in order to query identification data of the implantable medical device, in particular a model and/or firmware version, wherein the first control unit is further configured, based on the identification data, to read predetermined memory areas of the implantable medical device.

10. System of claim 9, wherein information contained in the predetermined memory areas of the implantable medical device comprises a current programming of the implantable medical device, trend data of the last 24 hours, last measured values from automatic tests, long-term trend data, a list of stored episodes, episode details of the episodes within the last 24 hours, patient data stored in the implant and/or a predetermined interval of live IEGM data.

11. System of claim 1, wherein the front-end communication unit is configured to send metadata from the implantable medical device to the back-end communication unit, wherein the back-end communication unit is configured to determine which memory areas the front-end communication unit is to query, and wherein the back-end communication unit is configured to transmit information on said memory areas to the front-end communication unit.

12. System of claim 1, wherein the cloud gateway is configured to distribute software updates to the cloud server, wherein the front-end communication unit is configured to query the cloud server for software updates.

13. System of claim 12, wherein the cloud server is configured to check on request of the front-end communication unit whether there is a distribution rule for the front-end communication unit, in particular based on a serial number and/or country/region of the front-end communication unit, wherein if a software update is available, the cloud server is configured to notify the front-end communication unit about the update.

14. Computer implemented method for communicating with an implantable medical device comprising the steps of:
reading out data from the implantable medical device by means of a front-end communication unit comprising a communication interface and a first control unit communicatively connected to the communication interface;
transferring the data read out by the communication interface to a back-end communication unit by means of the first control unit;
receiving the data transferred by the first control unit by means of a communication interface simulation unit comprised by the back-end communication unit;
simulating the communication interface by means of the communication interface simulation unit;
reading out data from the communication interface simulation unit by means of a programmer app unit comprised by the back-end communication unit; and
controlling the communication interface simulation unit and the programmer app unit by means of a second control unit comprised by the back-end communication unit in such a way that the data is saved, in particular as a report, in a storage unit communicatively connected to the second control unit and/or the back-end communication unit, said data being accessible by a health care provider.

15. Computer program with program code to perform the method of claim 14 when the computer program is executed on a computer.
